(19) United States
(12) Patent Application Publication
(10) Pub. No.: US 2007/0218111 A1
(43) Pub. Date: Sep. 20, 2007

(54) STRONTIUM COMPOSITIONS FOR BONES

(75) Inventors: Joel Ehrenkranz, Salt Lake City, UT (US); Nathalie Chevreau, Salt Lake City, UT (US); Cade Walker, Salt Lake City, UT (US)

Correspondence Address: HOLME ROBERTS & OWEN, LLP, 299 SOUTH MAIN, SUITE 1800, SALT LAKE CITY, UT, 84111, US

(73) Assignee: Western Holdings, LLC, Casper, WY (US)

(21) Appl. No.: 11/687,566

(22) Filed: Mar. 16, 2007

Related U.S. Application Data
(60) Provisional application No. 60/782,924, filed on Mar. 16, 2006.

(51) Int. Cl. A61K 33/24

(52) U.S. Cl. 424/439; 424/617; 514/167; 514/2; 514/23

(57) ABSTRACT

A composition of matter includes an effective amount of strontium carbonate for affecting bone metabolism. The composition is substantially free of calcium to avoid absorption competition in the digestive tract. The composition is also preferably substantially free of chelating agents, phosphate, phytic acid, oxalates, insoluble fibers, and non-strontium divalent cations or other materials that compete with strontium ions for absorption in the human digestive tract. Other ingredients are optionally added to increase strontium absorption or for their independent positive effective on bone health, including for example vitamin D and vitamin K, quercetin dihydrate, and hesperidin. The composition is preferably administered as a chewable food by blending with a chewable food matrix to form a palatable food that is readily consumed.

### CROSS-REFERENCE TO RELATED APPLICATIONS

This application claims the benefit of U.S. Provisional Patent Application No. 60/782,924, filed Mar. 16, 2006, entitled “STRONTIUM COMPOSITIONS FOR BONES,” which is incorporated herein by reference.

### BACKGROUND OF THE INVENTION

1. The Field of the Invention

The present invention relates to the fields of food compositions and dietary supplements. More particularly, the invention provides compositions and methods for the use of strontium carbonate in improving bone health.

2. The Relevant Technology

Bone involution is a normal part of aging. The age related loss of bone mass is known to occur universally with age in all human populations. As an individual ages, distinct changes take place in trabecular and cortical bone as well as bone marrow. The decrease in skeletal mass associated with increasing age is due to a series of aging-related changes. Both calcium intake and calcium absorption decrease with age. Parathyroid hormone levels increase; this results in a removal of calcium from bone. Serum 25-hydroxy vitamin D levels decline and this results in decreased gastrointestinal calcium absorption. Calcium resorption by the kidney becomes less efficient with age. Regardless of the cause, however, the age-related bone loss leads to a decrease in bone mineral density, a deterioration in the microscopic architecture of bone, and an increased susceptibility to osteoporosis.

Osteopenia, a reduction in bone mass that is considered part of the normal process of aging, is defined as a bone mineral density between 1 and 2.5 standard deviations below the mean for adults aged 30-40 years, while osteoporosis is arbitrarily defined as a bone mineral density 2.5 standard deviations below the mean for adults aged 30-40 years. The arbitrary statistical cutoffs used to label the inevitable loss of bone with age as osteopenia or osteoporosis do not parallel the clinical consequences of age-related bone loss, as the risk of fracture almost doubles for each standard deviation below peak bone mass. This linear increase in fracture risk is independent of bone density criteria for osteopenia or osteoporosis, as these categorizations are based upon statistical analyses of populations.

Osteoporosis is an inevitable accompaniment of aging. At age 65, x-ray comparison with a bone standard shows that 65% of women and 21% of men have osteoporosis. The US Food and Drug Administration defines osteoporosis “as a condition in which the bone mass per unit volume (density) of normally mineralized bone is reduced.” The FDA further characterizes osteoporosis as either type 1, which “affects women after menopause and results from an accelerated rate of bone loss due to factors related to menopause” or “type 2 (age related) osteoporosis involves both men and women over age 70 and is characterized by gradual (over several decades) loss of bone mass due to factors related to the aging process.”

While men lack the rapid phase of bone loss seen in women at menopause, they lose substantial amounts of bone as they age. The decrease in a man's bone mass from peak, at approximately age 30, to 80 years of age is between 13-18%. This appears to be due, after age 60, to an imbalance between bone formation and bone resorption. In men, aging is associated with a decline in the ability of bone forming cells to refill cavities excavated by the bone cells which are responsible for resorbing bone. The consequences of this are a gradual decrease in bone thickness as well as a logarithmic decrease in bone strength.

In summary, aging results in a loss of bone in both men and women. In fact, over the age of 65, more than half of all women and almost a quarter of all men will have some degree of osteoporosis. The more bone is lost, the weaker the bone becomes, and the more likely it is to fracture. Still, this age-related loss of bone is a natural biological process and does not indicate the presence of disease.

With aging, bones become brittle and are prone to lose structural integrity. The importance of exercise, calcium, and vitamin D in maintaining bone health are well-known standard practices to help maintain bone mass. Unfortunately, these everyday measures are oftentimes not enough to counteract the natural progression of bone loss due to aging. What is needed are improved methods to both retard the rate of age related bone loss and to replace bone loss due to the effects of the aging process on bone.

### BRIEF SUMMARY OF THE INVENTION

The present invention provides for new and improved compositions and methods of enhancing osteoblastic activity and inhibiting osteoclastic activity in bone. It is believed that the present invention not only provides compositions and methods for assisting in strengthening bones, but also for increasing adult stature by increasing epiphyseal bone growth, the healing of fractures, and treating metabolic bone disease.

The inventive compositions preferably include at least 100 mg of strontium carbonate in a formulation that is substantially free of calcium and other divalent cations or materials that compete with strontium through absorption pathways in the digestive tract. This increases the effectiveness of the dosage and helps protect the strontium from being passed through the digestive tract without being absorbed.

The strontium is preferably administered as an additive in a food such as a nutritional bar, dessert, confection or other food, preferably a chewable food.

Accordingly, a first example embodiment of the invention is a composition of matter including an effective amount of strontium carbonate for affecting bone metabolism and being substantially free of calcium. The composition can be in a dosage such as a beverage, a tablet, a capsule, a powder, or a chewable food.

In another example embodiment of the invention, a composition of matter includes a chewable food having an effective amount of strontium carbonate for affecting bone metabolism mixed with a chewable food-stuff matrix to form a chewable food. The chewable food is substantially lacking in calcium to prevent absorption interference in the digestive tract.

Another example embodiment of the invention is a method for improving bone health in a mammal, the method including administering an effective dose of strontium carbonate for improving bone health in a mammal in the absence of amounts of calcium that would interfere with the uptake of the strontium. The effective dose of strontium carbonate may be, for example, an amount sufficient to increase adult stature by increasing epiphyseal bone growth, sufficient for assisting in the healing of bone fractures, or sufficient for treating metabolic bone disease.

The example compositions preferably include sufficient strontium carbonate to provide at least about 100 mg of elemental strontium, more preferably from about 500 mg to about 1500 mg elemental strontium, still more preferably from about 600 mg to about 700 mg elemental strontium.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

In the following description, numerous specific details are set forth in order to provide a thorough understanding of the present invention. It will be obvious, however, to one skilled in the art that the present invention may be practiced without these specific details. In other instances, well-known aspects of strontium carbonate and nutritional supplements in general have not been described in particular detail in order to avoid unnecessarily obscuring the present invention.

The present invention provides for new and improved compositions and methods of introducing strontium into the human diet to enhance osteoblastic activity and inhibit osteoclastic activity in bone. It is believed that the present invention not only provides compositions and methods for assisting in strengthening bones, but also for increasing adult stature by increasing epiphyseal bone growth, the healing of fractures, and treating metabolic bone disease.

The inventive compositions preferably include at least 100 mg of strontium in the form of a neutral salt, such as carbonate, gluconate, lactate, chloride, citrate, etc., although the carbonate form is currently the most preferred. It is also currently preferred that a daily dosage of elemental strontium be administered in the range of about 600 to about 700 mg.

The strontium is preferably administered as an additive in a food, such as a nutritional bar, dessert, confection or other food. The inventive formulations can be quickly adsorbed into the human digestive tract and result in mitigating bone loss, treating metabolic bone disease, aiding in the healing of fractures, increasing stature in children, and the like.

The strontium is preferably administered in a formulation and environment free of calcium and other divalent cations or materials that compete with strontium through absorption pathways in the digestive tract. This increases the effectiveness of the dosage and helps protect the strontium from being passed through the digestive tract without being absorbed.

The skeleton is the body's organ of structural support. The skeleton is also a reservoir for essential body chemicals, including calcium, phosphate, and sodium. The skeleton consists primarily of bone, and bone consists of two forms: compact bone and trabecular bone. Compact bone is the dense outer shell of the skeleton, while trabecular bone consists of plates, rods, arches, and struts of bone contained within compact bone. Both compact and trabecular bone are made from protein and mineral. Bone is strong and hard because calcium phosphate crystals, hydroxyapatite, are deposited around the protein matrix.

Strontium can substitute for calcium in hydroxyapatite. Hydroxyapatite is similar to limestone, and bone can be thought of as stone crystals held together by proteins. Strontium exchanges with calcium in already formed hydroxyapatite crystals within bone mineral and is also incorporated directly into newly formed bone.

Bone is a living and dynamic material. With repeated use and over time, bone sustains microdamage and needs to be renewed or replaced. The process of replacing old and damaged bone is called remodeling. The bone cells responsible for remodeling are osteoblasts and osteoclasts. The process of remodeling is initiated by osteoclasts excavating out old and damaged bone, followed by osteoblasts filling in the excavated bone with new bone. Osteoclast activity followed by osteoblast activity in remodeling is tightly linked and is referred to as coupling. All the bone in the body is turned over every six years.

As mentioned above, strontium can replace calcium in the mineral crystals, hydroxyapatite, in bone. Strontium's effects on bone are to inhibit osteoclasts, that is to decrease the excavation of old bone, and to stimulate osteoblasts so that new bone is made. Strontium uncouples bone remodeling. No longer is bone made only following bone resorption; new bone is made independent of bone resorption. The net effect is an increase in bone. Strontium stimulates the process of bone building. The presence of strontium in bone correlates with bone compression strength.

Various bone building vitamins which do not interfere with the absorption of strontium can also be added to the foods. For example, compounds such as vitamin D3 (cholecalciferol) and/or vitamin K may be advantageously added. Vitamin D3 in this example aids in bone growth by increasing the absorption of strontium. Vitamin K1 is used by the body to form hydroxyproline, an amino acid used for bone formation.

Similarly, other natural products such as quercetin dihydrate and hesperidin can be added to enhance the formulation. Other conventional additives, stabilizers, pH adjusters, and the like can also added.

In addition to quercetin (and its glycone, rutin) and hesperidin, a number of other natural products can be included in a bone building food. Examples of such natural products include carnitine, acetyl carnitine, juniper berry oil, pine oil, dwarf pine oil, maritime pine bark extract, oil of rosemary, oil of sage, thujone, eucalyptol, camphor, borneol, menthol, a-pinene, b-pinene, thyrmol, bornylacetate, erbenol, cousmestrol, zearalenol, isolflavones, genistein, diadzin, humulone, phlorizin, horsetail extract, melatonin, and Cistanche salsa extract.

It is currently believed that the molecular effects of strontium on bone are mediated by the cellular calcium receptor. All mammals have a sensitive system to maintain blood calcium levels within a very narrow range. This system involves cell types in organs throughout the body, including bone, kidney, parathyroid, thyroid, and the small intestine. The cells in this system have receptors on their cell surface which respond to the extracellular calcium concentration and trigger specific cellular responses. Strontium mimics the effects of calcium on this cell membrane calcium sensing mechanism, but only when there is a sufficiently high concentration of strontium. Because 99% of the body's strontium is found in bone, a strontium concentration sufficient to activate the calcium receptor can only be achieved in bone.

Calcium, which is preferentially absorbed compared to strontium, interferes with strontium absorption, as do chelating agents, phytic acid, oxalates, fiber, and phosphate. As a result, ninety percent of absorbed strontium is typically excreted by the kidney; while 10% is excreted via the fecal stream. The kidney preferentially excretes strontium over calcium due to increased tubular resorption of the later cation. Accordingly, strontium is preferably administered in the absence of calcium. It is also preferred that strontium be administered in the absence of chelating agents, phytic acid, oxalates, phosphate, and other divalent cations. Also, it is preferable to ingest strontium on an empty stomach or separate from a meal in order to increase absorption of the strontium.

It may be useful to ingest substances along with strontium that facilitate strontium absorption. For example, suitable compositions and methods for administration of strontium may include the co-administration of lysine, lactose and/or arginine.

It is preferred that strontium carbonate be used having a purity of at least 99.4%, and that it does not include significant amounts of aluminum, arsenic, barium, cadmium, copper, magnesium, molybdenum, mercury, lead, iron, sodium, tin, sulfur, or vanadium.

By way of example, the inventive compositions can include a strontium carbonate blend which contains 59.3% by weight elemental strontium that has a minimum of impurities. One form of strontium is strontium carbonate. Each gram of strontium carbonate contains 593.5 mg of elemental strontium, so ingesting 1100 mg of strontium carbonate a day provides 653 mg of strontium.

The food compositions preferably include at least about 100 mg strontium carbonate. Alternatively, the food compositions preferably include from about 100 mg to about 1500 mg strontium carbonate, preferably from about 400 mg to about 1100 mg strontium carbonate, most preferably about 1000 mg strontium carbonate.

The food compositions can also include other ingredients. For example, the compositions can include from 10 to 900 international units vitamin D3 powder, more preferably from about 100 to 400 IU vitamin D3 powder. The food compositions can also preferably include from about 0.001 to about 10 mg vitamin K1 powder, and more preferably about 1 mg vitamin K1 powder. The compositions also preferably include from about 0.01 to about 1000 mg of one or more bioflavonoids such as quercetin or hesperidin, more preferably from about 0.05 to about 1000 mg, most preferably about 100 mg. The formulations may also include other active and inactive ingredients and/or may contain conventional excipients, carriers and diluents provided they do not interfere with the absorption of strontium.

The biological effects of strontium are independent of the type of strontium salt administered, as strontium is absorbed from the gut and resorbed from the kidney as a cation. The effects of strontium on the skeleton result solely from the strontium ion, as only the strontium is absorbed by the intestinal mucosa. The biological effects of strontium are independent of the anion with which strontium is administered. As a result, strontium salts in addition to strontium carbonate fall within the scope of various embodiments of the invention.

A variety of suitable forms of providing for human ingestion of the disclosed compositions can be used and provided. General examples include nutritional supplements, pharmaceutical preparations, and foods supplemented with the specified compositions of the invention. Many such packaging and administration forms for the compositions are known to those skilled in the art and are not discussed in detail herein to avoid obscuring the invention. For example, the formulation can be in the form of a pill, tablet, capsule, or similar dosage form.

However, single dose solid tablets or capsules having the herein disclosed quantities of strontium carbonate can be relatively large. This is due in part because of the density of strontium in strontium carbonate require a relatively large volume be consumed. Although the size of such tablets or capsules is not prohibitive for everyone, much of the consuming population, particularly aging women in highest need of the disclosed strontium supplements, has difficulty swallowing such large capsules and tablets. Further, even after a tablet or capsule is swallowed, it can cause discomfort as it travels down the esophagus.

Accordingly, a preferred method of delivering strontium carbonate is in the form of a food such as a nutritional bar, candy, or other confection for administering to a user. More particularly, a particularly preferred method of delivering strontium carbonate is in the form of a chewable food such as a confectionary having strontium carbonate mixed with a chewable food-stuff matrix.

In one embodiment, a chewable food-stuff matrix preferably includes one or more of a bulking agent (for example carbohydrates, corn syrup, fructose and/or maltodextrin), protein (for example casein, soy, and/or whey) a vegetable based oil, an emulsifier, a surfactant, a thickening agent (for example gum and/or cellulose) a natural flavoring additive, and a sweetener. For example, the chewable food-stuff matrix may include hydrogenated starch hydrolysate, corn syrup, sweetened condensed whole milk, cocoa, palm kernel oil, monoglyceride, diglycerides, natural flavors, sugar alcohol, and soy lecithin.

The amount of strontium carbonate in a chewable food may be as broad as 5% to about 60% by weight, but for purposes of consistency, texture, and taste it is more preferably from about 18% to about 21 by weight, most preferably about 20%. In addition to strontium carbonate, the chewable food may include other additives, for example vitamin D and vitamin K. Vitamin D may be, for example, added at from about 10 IU to about 800 IU, more preferably from about 100 IU to about 400 IU, in either case preferably limited by dietary guidelines such as the government's recommended daily allowances and desired intake. Similarly, vitamin K is preferably added at from about 1 mg to about 250 mg, more preferably from about 50 mg to about 90 mg, also as limited by dietary guidelines such as the government's recommended daily allowances and desired intake.

By way of example, a preferred chewable food can weigh between 1.5 grams to 20 grams, preferably about 5.5 grams.

In lieu of corn syrup, other suitable ingredients of a chewable food matrix can be added for viscoelasticity and may include, for example, sucrose, polydextrose, maltodextrin, rice syrup, fructose syrup, or a hydrogenated glucose syrup (HSH) or the like and mixtures thereof in combination with a humectant such as glycerine, propylene glycol or the like. As a vegetable oil, palm kernel oil is particularly preferred, but other suitable examples may include coconut oil, partially hydrogenated soybean oil, partially hydrogenated cottonseed oil or the like or any other vegetable oil or any fat derived emulsifier. Emulsifiers may also be utilized in the composition, for example lecithin or the like.

Additional materials which can be incorporated into a chewable food to enhance its appearance, taste, and consistency include, for example, flavors, sweeteners, and colorants. Flavors may be chosen from natural and synthetic flavoring liquids. An illustrative list of such agents includes volatile oils, synthetic flavor oils, flavoring aromatics, oils, liquids, oleoresins or organic extracts and combination thereof. Other useful flavorings and mixtures thereof are known in the art, including specific chemicals as well as other flavorings such as whole and partial fruits and nuts, peanut butter, candy bits, chocolate chips, bran flakes, etc.

Sweeteners may also be added to the chewable foods of the invention. These enhance the flavor and impart a palatable sweetness to the food. The sweeteners may be chosen from one or more of the following non-limiting list in addition to other ingredients having a sweetening effect. Example sweeteners include: glucose, dextrose, invert sugar, fructose, and mixtures thereof saccharin and its various salts such as the sodium salt, dipeptide sweeteners such as aspartame, dihydrochalcone compounds, glycyrrhizin, Stevia Rebaudiana (Stevioside), chloro derivatives of sucrose such as sucralose, sugar alcohols such as sorbitol, mannitol, xylitol, and natural sweeteners such as lo han guo, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof. Other sweeteners may also be used as are known in the art. The sweeteners are preferably added in amounts equal to about 0-10% of the composition, and preferably about 0.1-5%.

Surfactants or emulsifiers may also be included in the composition of the invention. These may be any food grade emulsifying material, for example, lecithin or other phospholipid material, monoglycerides and/or diglycerides, and mixtures thereof in amounts of from about 0-3%, more desirably about 0.1-1%.

Other examples of suitable dosage forms include supplemented foods, liquids, powders, syrups, and the like as are known in the art.

The following example of the invention is shown by way of illustration only.

### EXAMPLES

### Example 1

A single dose composition was prepared according to the following table.

| | Raw Material | Raw | |
|---|---|---|---|
| Raw Ingredients | mg/Serving | mg/cap | % DV |
| Strontium Carbonate (59.3% | 1011.80 | 1011.80 | * |
| strontium); purity greater or equal | | | |
| to 99.4% | | | |
| Vitamin D3 Powder | 4.00 | 4.00 | 100% |
| (cholecalciferol) 100,000 IU/g | | | |
| Vitamin K1 Powder (5% vit. K1) | 1.60 | 1.60 | 100% |
| Quercetin Dihydrate | 0.10 | 0.10 | * |
| Hesperidin | 0.10 | 0.10 | * |
| Formula Total | 1017.60 | | |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition of matter, comprising an effective amount of strontium carbonate for affecting bone metabolism, and being substantially free of calcium.

2. The composition of claim 1, wherein the composition comprises sufficient strontium carbonate to provide at least about 100 mg of elemental strontium.

3. The composition of claim 1, wherein the composition comprises sufficient strontium carbonate to provide from about 500 mg to about 1500 mg elemental strontium.

4. The composition of claim 1, further comprising the addition of an effective amount of one or more compounds selected from the group consisting of lysine, lactose, and arginine to facilitate strontium absorption.

5. The composition of claim 1, which is substantially free of chelating agents.

6. The composition of claim 1, which is substantially free of phosphate, phytic acid, oxalates, and insoluble fibers.

7. The composition of claim 1, which is substantially free of non-strontium divalent cations or other materials that compete with strontium ions for absorption in the human digestive tract.

8. The composition of claim 1, further comprising from about 100 international units to about 800 international units of vitamin D3.

9. The composition of claim 1, further comprising from about 0.001 grams to about 10 grams of vitamin K.

10. The composition of claim 1, further comprising:
0.1 mg to 1000 mg quercetin dihydrate; and
0.1 mg to about 1000 mg hesperidin.

11. The composition of claim 1, comprising a dosage form selected from the group consisting of a beverage, a tablet, a capsule, a powder, and a chewable food.

12. A composition of matter, comprising:
a chewable food-stuff matrix substantially lacking in calcium, the chewable food-stuff matrix comprising an effective amount of strontium carbonate for affecting bone metabolism.

13. The composition of claim 12, which is substantially free of chelating agents, phosphate, phytic acid, oxalates, insoluble fibers, and non-strontium divalent cations or other materials that compete with strontium ions for absorption in the human digestive tract.

14. The composition of claim 12 further comprising:
an effective amount of vitamin D3 for bone building activity; and
an effective amount of at least one of quercetin dihydrate and hesperidin for bone building activity.

15. The composition of claim 14 wherein the vitamin D3 comprises about 100 and 800 international units.

16. The composition of claim 14, further comprising from about 0.001 grams to about 10 grams vitamin K.

17. The composition of claim 12, further comprising:
from about 0.1 mg to about 1000 mg quercetin dihydrate; and
from about 0.1 mg to about 1000 mg hesperidin.

18. The composition of claim 12, wherein the chewable food-stuff matrix comprises a bulking agent, a vegetable based oil, a natural flavoring additive, a sweetener, and at least one of an emulsifier and a surfactant.

19. The composition of claim 12, wherein the strontium carbonate is from about 18 to about 21% by weight of the composition.

20. The composition of claim 12, wherein the composition comprises sufficient strontium carbonate to provide at least about 100 mg of elemental strontium.

21. The composition of claim 12, wherein the composition comprises an amount of strontium carbonate to provide from about 500 mg to about 1500 mg strontium carbonate.

22. The composition of claim 12, wherein the composition comprises an amount of strontium carbonate to provide from about 600 mg to about 700 mg of elemental strontium.

23. The composition of claim 12, the composition having a weight of from about 1.5 grams to about 20 grams.

24. The composition of claim 12, further comprising the an effective amount of one or more compounds selected from the group consisting of lysine, lactose, and arginine to facilitate strontium absorption.

25. A method for improving bone health in a mammal, the method comprising administering an effective dose of strontium carbonate, in the absence of amounts of calcium that would interfere with the uptake of the strontium, for improving bone health in a mammal.

26. The method of claim 25, wherein the effective dose of strontium carbonate comprises an effective dose of strontium carbonate to increase adult stature by increasing epiphyseal bone growth.

27. The method of claim 25, wherein the effective dose of strontium carbonate comprises an effective dose of strontium carbonate for assisting in the healing of bone fractures.

28. The method of claim 25, wherein the effective dose of strontium carbonate comprises an effective dose of strontium carbonate for treating metabolic bone disease, in the absence of amounts of calcium that would interfere with the uptake of the strontium.
